(19) United States
(12) Patent Application Publication
(10) Pub. No.: US 2016/0166723 A1
(43) Pub. Date: Jun. 16, 2016

(54) DEVICE AND METHOD FOR DISPENSING A DISINFECTING AGENT

(71) Applicant: MCC Global Laboratories, Inc., Middleton, MA (US)

(72) Inventor: Loutfi Kachouh, Juvignac (FR)

(73) Assignee: MCC Global Laboratories, Inc.

(21) Appl. No.: 14/967,682

(22) Filed: Dec. 14, 2015

Related U.S. Application Data
(60) Provisional application No. 62/092,356, filed on Dec. 16, 2014.

(51) Int. Cl. A61L 9/14; A61L 2/22

(52) U.S. Cl. CPC A61L 9/14; A61L 2/22

(57) ABSTRACT

A device for dispensing a disinfecting agent includes a container made of a non-oxidizing, non-elastic material of a predetermined volume configured to store a predetermined amount of a disinfecting agent and a predetermined propellant at a predetermined pressure. A diffusion device coupled to the container is configured to nebulize a fog of the disinfecting agent into a space having a predetermined volume and pathogens therein. The container and diffusion device and a combination of predetermined volume of the container, the predetermined amount of the disinfecting agent, and the predetermined pressure of the propellant are configured for the space such that the fog of disinfecting agent disinfects the pathogens in the air and surfaces of the space.

### RELATED APPLICATIONS

This application claims benefit of and priority to U.S. Provisional Application Ser. No. 62/092,356 filed Dec. 16, 2014 under 35 U.S.C. §§119, 120, 363, 365, and 37 C.F.R. §1.55 and §1.78, which is incorporated herein by this reference.

### FIELD OF THE INVENTION

The subject invention relates to a device and method for dispensing a disinfecting agent.

### BACKGROUND OF THE INVENTION

Various spaces in healthcare facilities, clinics, laboratories, research facilities, pharmaceutical manufacturing facilities, veterinarian facilities, and the like, often require disinfecting to prevent spread of infection and contamination. For example, the spread of infection in healthcare facilities has become the focus of increased regulation that is no longer tolerated without severe financial penalties. In order to minimize the cost associated with laborious, conventional, manual disinfecting methods, “no-touch” cleaning robotics have been developed which may use ultra-violet light or a fog of disinfecting agent to disinfect a desired space.

Conventional devices for dispensing a fog of disinfecting agent to disinfect a space, such as the operating room of a hospital or any similar type space of the facilities discussed above, are typically large, bulky, complicated, and expensive devices. Such conventional devices often require extensive training, maintenance and periodic calibration for certification before they can be utilized. The operator may also need to determine the size of the room, the fogging time, the amount of disinfectant to be used, and the like. Additionally, such conventional devices may require electrical and computer cables which plug into outlets in the wall of the space to be disinfected. Thus, conventional devices for dispensing a fog of disinfecting agent are typically cumbersome and often difficult to use. Additionally, such conventional devices often require regular maintenance and calibration to regulate the quantity of disinfectant composition to be defused.

### BRIEF SUMMARY OF THE INVENTION

In one aspect, a device for dispensing a disinfecting agent is featured. The device includes a container made of a non-oxidizing, non elastic material of a predetermined volume configured to store a predetermined amount of a disinfecting agent and a predetermined propellant at a predetermined pressure. A diffusion device coupled to the container is configured to nebulize a fog of the disinfecting agent into a space having a predetermined volume and pathogens therein. The container and diffusion device and a combination of predetermined volume of the container, the predetermined amount of the disinfecting agent, and the predetermined pressure of the propellant are configured for the space such that the fog of disinfecting agent disinfects the pathogens in the air and surfaces of the space.

In one embodiment, the predetermined volume of the container may be in the range of 125 ml to about 2500 ml amount of disinfecting agent in the range of about 12 ml to about 20 ml for each 1 m³ of the predetermined space, the propellant may include a mixture of nitrogen and carbon dioxide, the predetermined pressure may be in the range of about 2 bar to about 15 bar, and the space having the predetermined volume may include a volume in the range of about 2 m³ to about 100 m³. The predetermined volume of the container may be about 125 ml, the predetermined amount of the disinfecting agent may be about 24 ml, the predetermined pressure may be in the range of about 2 bar to about 3 bar, and the space having the predetermined volume may be about 2 m³. The predetermined volume of the container may be about 210 ml, the predetermined amount of the disinfecting agent may be about 60 ml, the predetermined pressure may be in the range of about 3 bar to about 4 bar, and the space having the predetermined volume may be about 5 m³. The predetermined volume of the container may be about 520 ml, the predetermined amount of the disinfecting agent may be about 120 ml, the predetermined pressure may be in the range of about 3 bar, to about 5 bar, and the space having the predetermined volume may be about 10 m³. The predetermined size of the container may be about 800 ml, the predetermined amount of the disinfecting agent may be about 300 ml, the predetermined pressure may be in the range of about 4 bar to about 6 bar, and the space having the predetermined volume may be about 25 m³. The predetermined size of the container may be about 1500 ml, the predetermined amount of the disinfecting agent may be about 600 ml, the predetermined pressure may be in the range of about 7 bar to about 10 bar, and the space having the predetermined volume may be about 50 m³. The predetermined size of the container may be about 2500 ml, the predetermined amount of the disinfecting agent may be about 1200 ml, the predetermined pressure may be in the range of about 8 bar to about 14 bar, and the space having the predetermined volume may be about 100 m³. The container and diffusion device and a combination of predetermined volume of the container, the predetermined amount of the disinfecting agent, and the predetermined pressure of the propellant may be configured for a desired space. The disinfecting agent may include a balanced mixture of acetic acid (CH₃CO₂H), hydrogen peroxide (H₂O₂), peracetic acid (CH₃CO₃H), and water (H₂O). The disinfecting agent may include a mixture of about 5% peracetic acid (CH₃CO₃H), about 20% hydrogen peroxide (H₂O₂), about 5% acetic acid (CH₃CO₂H), and about 70% water (H₂O). The mixture of nitrogen and carbon dioxide may include about 67% nitrogen and about 33% carbon dioxide. The mixture of nitrogen and carbon dioxide may include about 75% nitrogen and about 25% carbon dioxide. The fog of disinfecting agent may disinfect at least 10⁶ pathogens in the air and surfaces of the space. The fog of disinfecting agent may disinfect at least 10⁵ pathogens in the air and surfaces of the space. The fog of disinfecting agent may disinfect at least 10⁴ pathogens in the air and surfaces of the space. The system may include a bag disposed inside the container configured to store the disinfecting agent therein. The bag may be configured to prevent the disinfecting agent from reacting with the container.

In another aspect, a device for dispensing a disinfecting agent is featured. The device includes a container made of a non-oxidizing, non elastic material of a predetermined volume in the range of about 125 ml to about 2500 ml configured to store a predetermined amount of a disinfecting agent in the range of about 12 ml to about 20 ml for each 1 m³ of a predetermined space and a propellant including carbon dioxide and nitrogen at a predetermined pressure in the range of about 2 bar to about 14 bar. A diffusion device is coupled to the container and configured to nebulize a fog of the disinfecting agent into a space having a predetermined volume in the range of about 2 m³ to about 100 m³ and pathogens therein. The container and the diffusion device and a combination of predetermined volume of the container, the predetermined amount of the disinfecting agent, and the predetermined pressure of the propellant are configured for the space such that the fog of disinfecting agent disinfects the pathogens in the air and surfaces of the space.

In one aspect, a method for dispensing a disinfecting agent is featured. The method includes providing a container made of a non-oxidizing, non elastic material of a predetermined volume configured to store a predetermined amount of a disinfecting agent and a predetermined propellant at a predetermined pressure. It provides a diffusion device coupled to the device to nebulize a fog of disinfecting agent into a space having a predetermined volume and pathogens therein. Choose a combination of the predetermined volume of the container, the predetermined amount of disinfecting agent, and the predetermined pressure of the propellant for the space such that the fog of disinfecting agent disinfects the pathogens in the space. Place the container and the diffusion device in the space and activating the diffusion device to dispense the fog of disinfecting agent into the space to effectively disinfect pathogens in the air and surfaces of the space.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a three-dimensional front-view of one conventional device for dispensing a fog of a disinfecting agent;

FIG. 2 is a three-dimensional front-view of another conventional device for dispensing a fog of a disinfecting agent;

FIG. 3 is a schematic front-view of one embodiment of the device for dispensing a disinfecting agent of this invention;

FIG. 4 is a schematic view of another embodiment of the device for dispensing a disinfecting agent of this invention;

FIG. 5 is a schematic front-view of another embodiment of the device for dispensing a disinfecting agent of this invention;

FIG. 6 is a schematic front-view of another embodiment of the device for dispensing a disinfecting agent of this invention;

FIG. 7 is a schematic front-view of yet another embodiment of the device for dispensing a disinfecting agent of this invention;

FIG. 8 is a schematic front-view of yet another embodiment of the device for dispensing a disinfecting agent of this invention;

FIGS. 9A, 9B, 9C, 9D, and 9E are schematic diagrams showing in further detail one embodiment of the diffusion device shown in FIGS. 3-8; and

FIG. 10 shows schematic diagrams of one embodiment of bag on value which may be utilized by the device for dispensing a disinfecting agent shown in one or more of FIGS. 3-9E.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

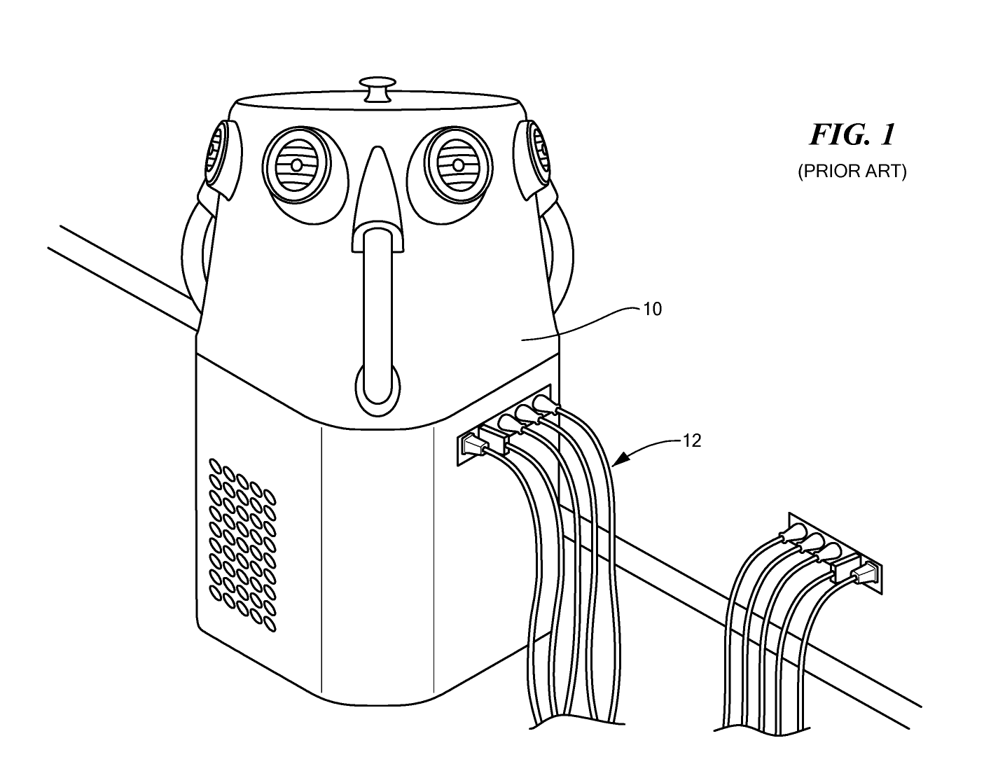

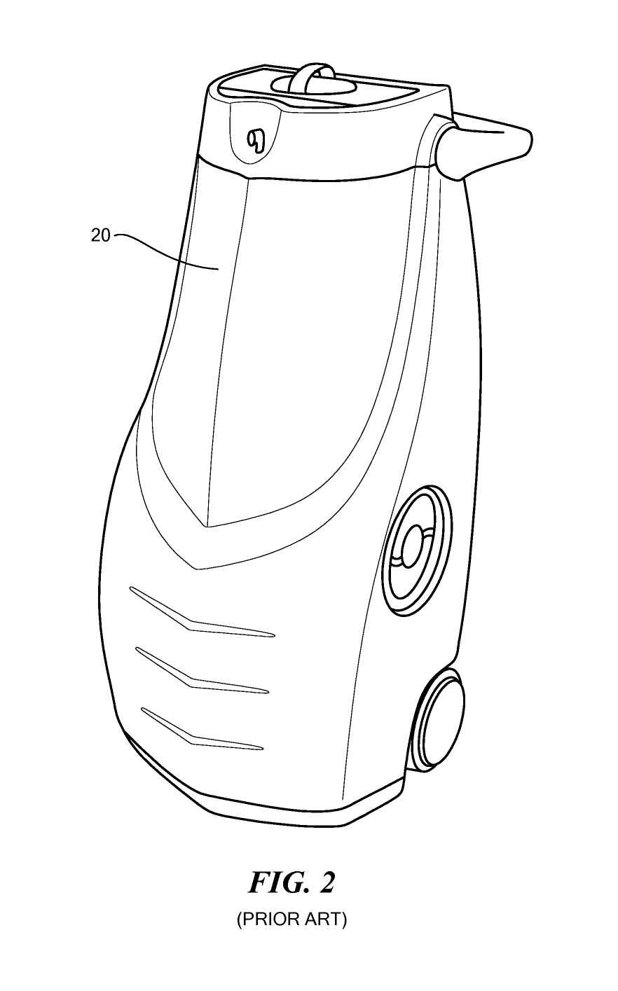

As discussed in the Background section, conventional devices for dispensing a disinfecting agent are large, bulky, complicated, expensive devices which may be cumbersome and difficult to use. For example, conventional device 10, FIG. 1, for dispensing a fog of disinfecting agent is a large, bulky and expensive device which requires extensive operator training, regular calibration and cleaning. Device 10 also utilizes electrical and computer cabling which need to be plugged into outlets, e.g., as indicated at 12. Device 20, FIG. 2, shows another example of a large, complicated, and expensive conventional device for dispensing a fog of disinfecting agent.

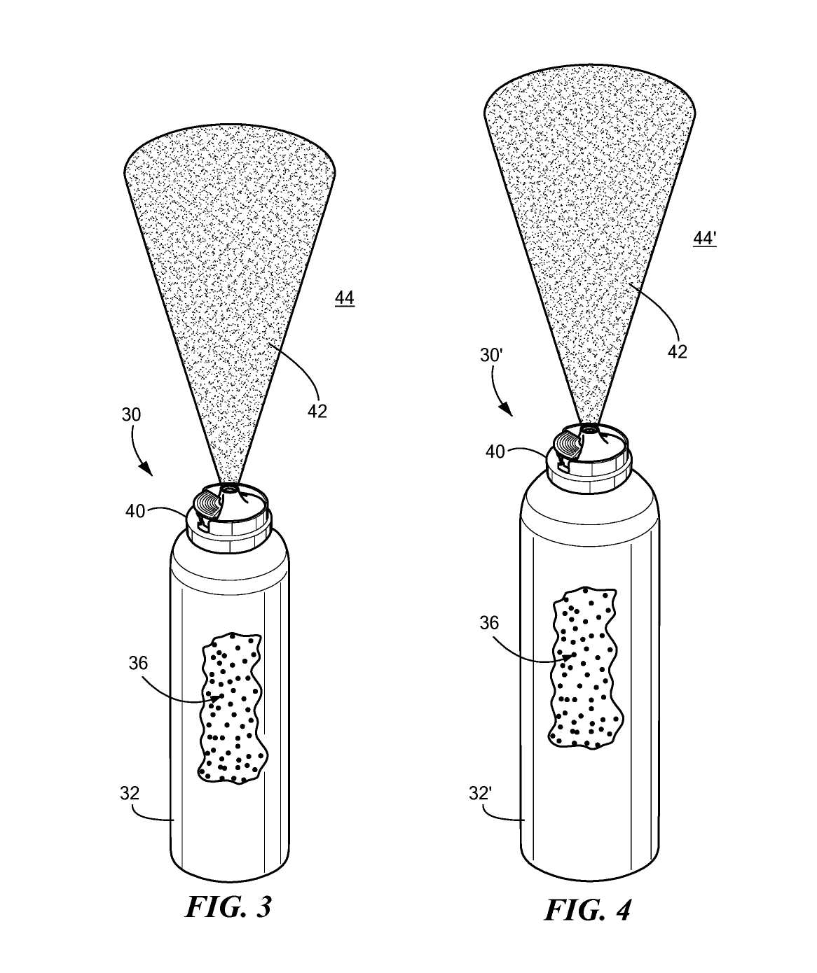

There is shown in FIG. 3, one embodiment of device 30 for dispensing a disinfecting agent. Device 30 includes container 32 made of a non-oxidizing, non-elastic material, such as metal, alloy, glass, or similar type material. Container 32 has a predetermined volume and is configured to store a predetermined amount of disinfecting agent therein, exemplarily indicated at 36, and a predetermined propellant at a predetermined pressure, as discussed in detail below. Device 30 also includes diffusion device 40 coupled to container 32 configured to nebulize fog 42 of the disinfecting agent into space 44 having a predetermined volume and pathogens therein. Container 32 and diffusion device 40 and a combination of the predetermined volume of container 32, the predetermined amount of disinfecting agent 36, and the predetermined pressure of the propellant are configured for space 44 such that fog 42 of disinfecting agent disinfects pathogens in the air and surfaces of the space 44 having a predetermined volume. In one example, fog 42, effectively disinfects pathogens, such as pathogenic microorganisms, bacteria, viruses, prions, fungus, and the like, in the air and surfaces of space 44.

In one embodiment, the predetermined volume of the container is in the range of about 125 ml to about 2,500 ml, the amount of disinfecting agent is in the range of about 12 ml to about 20 ml for each 1 m³ of the predetermined space, the predetermined pressure is in the range of about 2 bar to about 14 bar, and predetermined space having the predetermined volume includes a volume in the range of about 2 m³ to about 100 m³.

For the example shown in FIG. 3, the predetermined volume of container is about 125 ml, the predetermined amount of disinfecting agent is about 24 ml, and the predetermined pressure is in the range of about 2 bar to about 3 bar, and space 44 has a volume of about 2 m³. See Table 1 below:

TABLE 1
Various combinations of examples of the volume of the container, the
volume of disinfecting agent, the composition of the propellant, the
pressure in the container, and the volume of the space disinfected.

| | | | Volume of | | | |
|---|---|---|---|---|---|---|
| | | | Disinfecting | | | |
| | Room/space | Container | Agent in | | | Pressure in the |
| | volume | Volume | Container | Disinfecting | | container |
| | m³ | ml | ml | Agent | Propellant | bar |
| 1 | 2 | 125 | 24 | Peracetic acid, | Nitrogen + | 2-3 |
| 2 | 5 | 210 | 60 | acetic acid, | CO₂ | 3-4 |
| 3 | 10 | 520 | 120 | hydrogen | | 3-5 |
| 4 | 25 | 800 | 300 | peroxide, and | | 4-6 |
| 5 | 50 | 1500 | 600 | water | | 7-10 |
| 6 | 100 | 2500 | 1200 | | | 8-14 |

In another example, device 30′, FIG. 4, where like parts have been given like numbers, includes container 32′ having a volume of about 210 ml. In this example, the predetermined amount of disinfecting agent is about 60 ml, the predetermined pressure in container 32′ is in the range of about 3 bar to 4 bar, and space 44′ has a volume of about 5 m³. See Table 1 above.

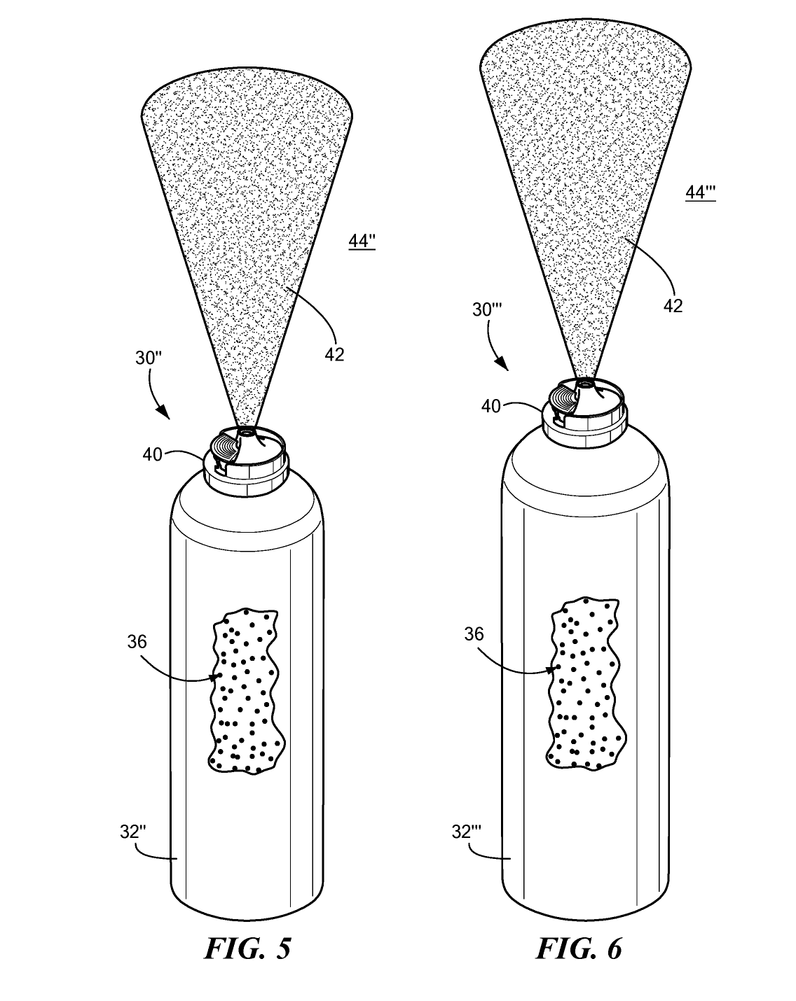

In yet another example, device 30″, FIG. 5, where like parts have been given like numbers, includes container 32″ having a volume of about 520 ml. In this example, the predetermined amount of disinfecting agent is about 120 ml, the predetermined pressure is in the range of about 3 bar to about 5 bar, and space 44″ has a volume of about 10 m³. See Table 1 above.

In yet another example, device 30′″, FIG. 6, where like parts have been given like numbers, includes container 32′″ having a volume of about 800 mL In this example, the predetermined amount of disinfecting agent is about 300 ml, the predetermined pressure is in the range of about 4 bar to about 6 bar, and space 44′″ has a volume of about 25 m³. See Table 1 above.

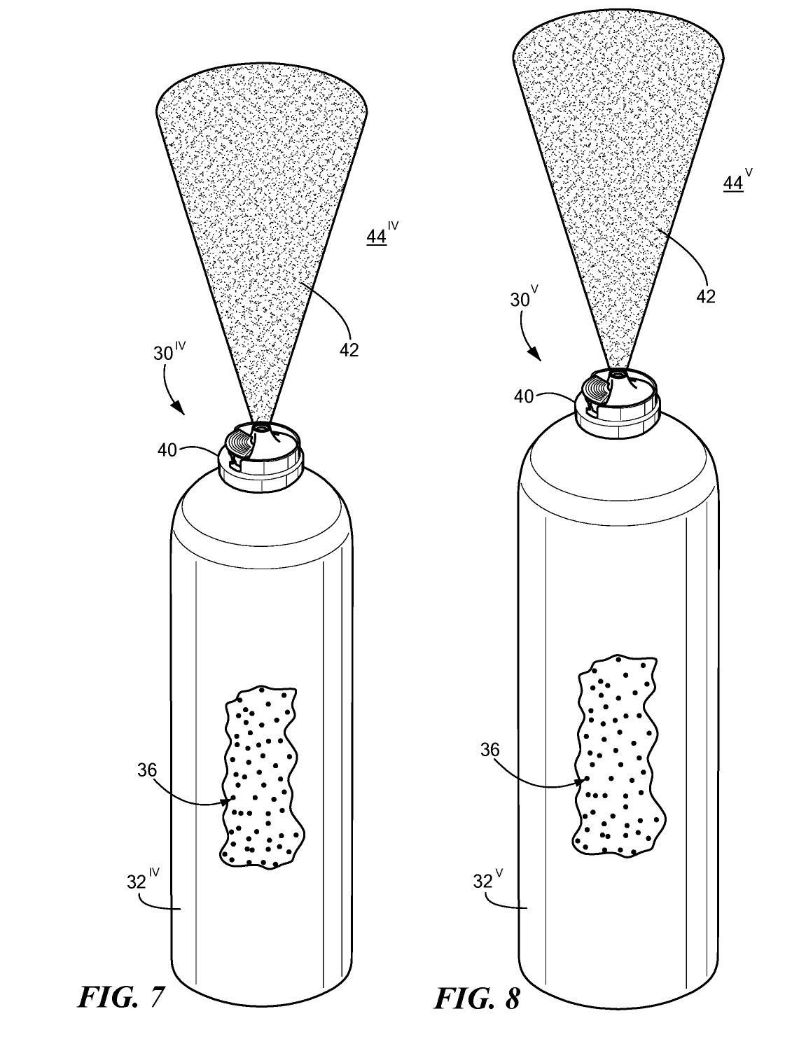

In yet another example, device 30^{IV}, FIG. 7, where like parts have been given like numbers, includes container 32^{IV} having a volume of about 1500 ml. In this example, the predetermined amount of disinfecting agent is about 600 ml, the predetermined pressure is in the range of about 7 bar to about 10 bar, and space 44^{IV} has a volume of about 50 m³. See Table 1 above.

In yet another example, device 30^{V}, FIG. 8, where like parts have been given like numbers, includes container 32^{V} having a volume of about 2500 ml. In this example, the predetermined amount of disinfecting agent is about 1200 ml, the predetermined pressure is in the range of about 8 bar to about 14 bar, and space 44^{V} has a volume of about 100 m³. See Table 1 above.

Although as shown in FIGS. 3-8, container 32, 32′, 32″, 32′″, 32^{IV}, 32^{V}, of device 30, 30′, 30″, 30′″, 30^{IV}, 30^{V} is designed for space 44, 44′, 44″, 44′″, 44^{IV,} and 44^{V} having a volume of 2 m³, 5 m³, 10 m³, 25 m³, 50 m³, and 100 m³, respectively, in other examples, device 30 may be custom tailored for a space of any desired volume. In this design, the size of container 32, the amount of disinfecting agent of in the range of about 12 ml to about 20 ml for each 1 m³ of the desired space, and the pressure of the propellant are adjusted for the desired space, e.g. the space having a volume less than about 2 m³ the space having a volume between various examples shown in Table 1 above, e.g., between 2 m³ and 5m³, 5 m³ and 10 m³, 10 m³ and 25 m³, 25 m³ and 50 m³ or 50 m³ and 100 m³ or the space having a volume greater than 100 m³.

In one example, a disinfecting agent includes a balanced mixture of acetic acid (CH₃CO₂H), hydrogen peroxide (H₂O₂), peracetic acid (CH₃CO₃H), and water. In one example, the disinfecting agent includes a mixture of about 5% peracetic acid, (CH₃CO₃H), about 20% hydrogen peroxide (H₂O₂), about 5% acetic acid (CH₃CO₂H), and about 70% water. (H₂O). Preferably, the propellant includes a mixture of nitrogen and carbon dioxide. In one example, the mixture of nitrogen and carbon dioxide is about 75% nitrogen and about 25% carbon dioxide. In another example, the mixture of nitrogen and carbon dioxide includes about 67% nitrogen and about 33% carbon dioxide.

In operation, one of device 30, 30′, 30″, 30′″, 30^{IV}, 30^{V}, FIGS. 3-8, is chosen for the appropriate space for healthcare facilities, clinics, laboratories, research facilities, pharmaceutical manufacturing facilities, veterinarian facilities, and the like, as discussed above and shown in Table 1. For example, device 30 may be chosen for space having a volume of 2 m³, e.g., a Bio Safety Cabinet (BSC Type II), device 30′ may be chosen for a space having a volume of 5 m³, e.g., a Vertical Laminar Air Flow Workstation (VLAFW), device 30″ may be chosen for a space having a volume of 10 m³, e.g., a Weighing Station (WS), device 30′″ may be chosen for a space having a volume of 25 m³ e.g., a bone marrow transplant room or for a space having a volume of 50 m³, e.g., an Intensive Care (IC) room, and device 30^{IV}, or 30^{V} may be chosen for the space having a volume of 100 m³, e.g., a Surgery Room (SR). The aforementioned spaces are for exemplary purposes only. Other spaces for healthcare facilities, clinics, laboratories, research facilities, pharmaceutical manufacturing facilities, veterinarian facilities, and the like, which may be disinfected by the device for dispensing a disinfecting agent of one or more embodiments of this invention are known to those skilled in the art. After the determination of the appropriate device 30-30^{V} needed for the space, the chosen device 30-30^{V} is then placed in the space, diffusion device 40 is activated (discussed in further detail below) and the user leaves space 44. Nebulized fog 42 then permeates the entire room until device 30-30^{V} is empty, e.g., about 1 minute for device 30, about 1.5 minutes for device 30′, about 2 minutes for device 30″, about 3 minute for device 30′″, about 4 minutes for device 30^{IV}, and about 5 minutes for device 30^{V}. After the appropriate amount of time, the pathogens in the air and surfaces of space 44-44^{V} are effectively disinfected.

The result is device 30-30^{V} provides an inexpensive, easy to use device that effectively disinfects the air and surfaces of space 44-44^{V} without the need for a large, bulky and expensive fogging device as discussed in the Background section above.

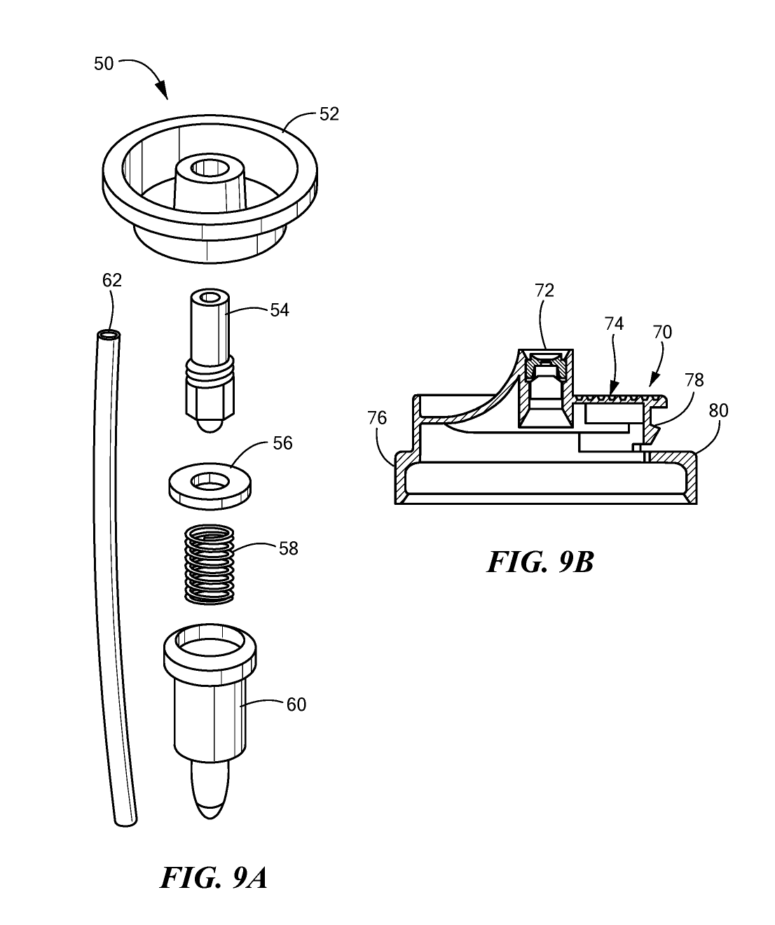

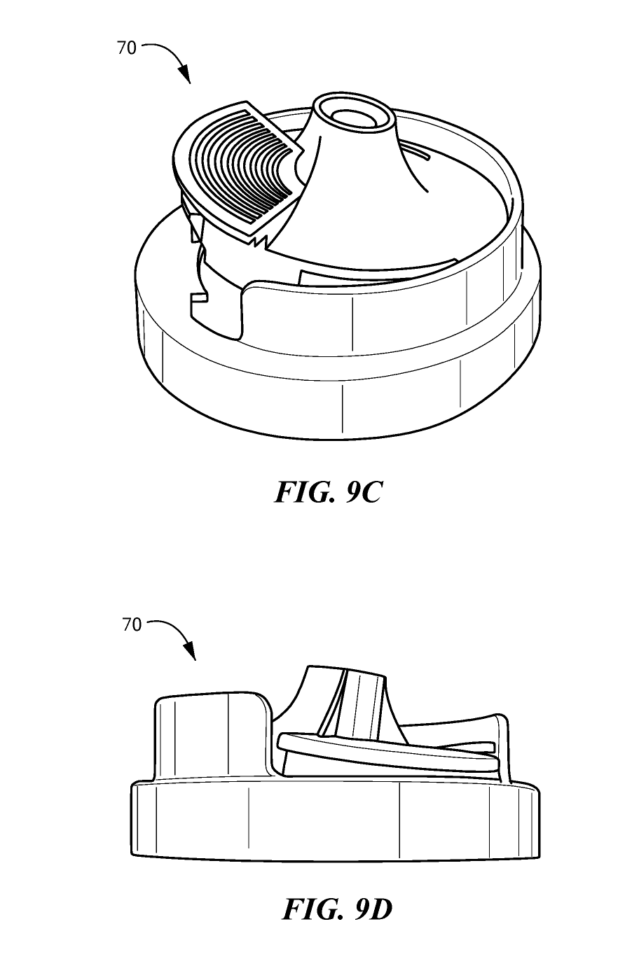

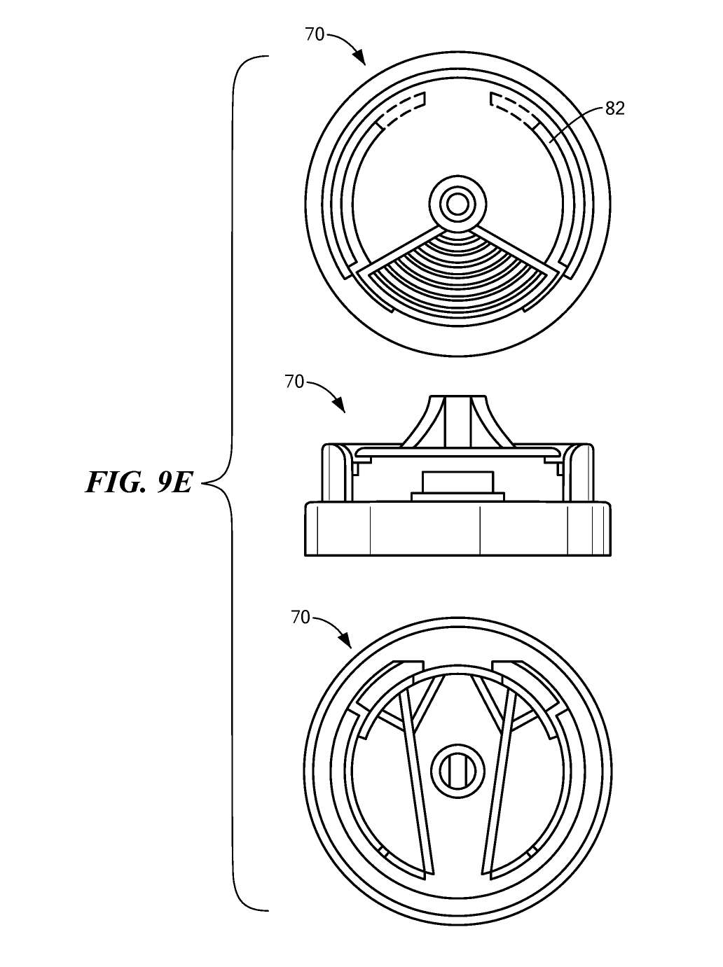

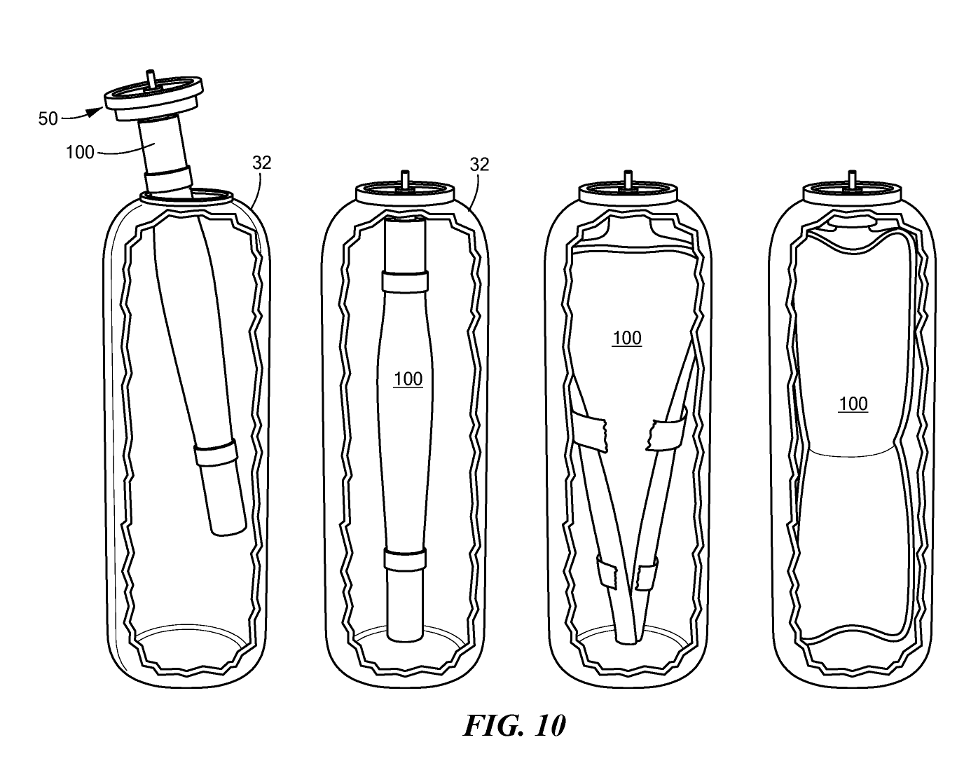

In one embodiment, diffusing device 40, FIGS. 3-7, may include diffusion subsystem or valve assembly 50, FIG. 9A, which is coupled to container 32, 32′, 32″, 32′″, 32^{IV}, FIGS. 3-7, as shown. Diffusion subsystem 50 includes mounting cap 52, stem 54, steam gasket 56, spring 58, body 60, and dip tube 62. In one example, diffusion subsystem 50 is available from Coster, Corso Como 15 20154 Milano, Italy.

Diffusion device 40 also preferably includes diffusion head or actuator 70, FIG. 9B. In one design, diffusion head or actuator 70 is available from Coster, Corso Como 15 20154 Milano, Italy. In one example, diffusion head 70 includes nozzle 72 and pushbutton 74 attached to body 76 and includes hook 78 which engages protruding member 80 when depressed such that diffusion subsystem 50 of diffusion device 40 dispenses fog 42, FIGS. 3-8 of disinfecting agent in container 32, 32′, 32″, 32′″, 32^{IV}, 32^{V} into space 44, 44′, 44″, 44′″, 44^{IV}, 44^{V} to disinfect or destroy the pathogens therein. FIG. 9C shows an example of diffusion head 70 with push button 74 in the release position. FIG. 9D shows an example of pushbutton 74 depressed and in the lock position. In one embodiment, diffusion head 70 may be configured as shown in FIG. 9E and preferably includes a plurality of slits 82 which vertically dispense fog 42, FIGS. 3-7, of disinfecting agent 36 into the space 44, as discussed above with reference to one or more of FIGS. 3-7.

In one example, device 30, 30′, 30″, 30′″, 30^{IV}, 30^{V}, FIGS. 3-8, can disinfect at least 10⁶ pathogens in the air and surfaces of space 44, commonly known as Log 6 disinfection. This is preferably for use in hospital operating rooms, and the like, which require a Log 6 sterilization. In other examples, device 30-30^{V} may disinfect at least 10⁵ pathogens in the air and surfaces of space 44, commonly known as Log 5. This may be used for intensive care, post-operative areas, quarantined areas, and the like. In yet another example, device 30-30^{V}, FIGS. 3-8, may disinfect at least 10⁴ pathogens in the air and surfaces of space 44, commonly known as Log 4, e.g., for use in patient rooms, ambulances, and the like. As known by those skilled in the art, testing for Log 6, Log 5, and Log 4, typically requires the use of indicators which have the desired number of pathogens thereon. For example, an indicator having at least 10⁶ (Log 6) pathogens may be placed in various locations in the predetermined space and then tested to determine if indeed Log 6, Log 5 or Log 4 pathogens in the air and surfaces of the space were effectively disinfected.

In one embodiment, dispensing device 30, 30′, 30″, 30′″, 30^{IV}, and 30^{V}, FIGS. 3-8, may include bag 100, FIG. 10, where like parts have been given like numbers, coupled to dispensing subsystem 50 and disposed inside container 32 as shown. In one example, bag 100 may be ball-on-valve, available from Summit Packing (Manchester, N.H.). In this example, the disinfecting agent is disposed inside bag 100 and the propellant at the desired pressure is disposed outside bag 100. Bag 100 is configured to prevent the disinfecting agent from reacting with container 32. The propellant outside bag 100 causes fog 42, FIGS. 3-8 of disinfecting agent 36 to be dispensed into space 44-44^{V}. See Table 1. In this example, the propellant is only nitrogen. Preferably, diffusion device 40, FIGS. 3-8, is integrated with bag 100, FIG. 10.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A device for dispensing a disinfecting agent, the device comprising:
a container made of a non-oxidizing, non-elastic material of a predetermined volume configured to store a predetermined amount of a disinfecting agent and a predetermined propellant at a predetermined pressure;
a diffusion device coupled to the container configured to nebulize a fog of the disinfecting agent into a space having a predetermined volume and pathogens therein; and
wherein the container and diffusion device and a combination of predetermined volume of the container, the predetermined amount of the disinfecting agent, and the predetermined pressure of the propellant are configured for the space such that the fog of disinfecting agent disinfects the pathogens in the air and surfaces of the space.

2. The system of claim 1 in which the predetermined volume of the container is in the range of 125 ml to about 2500 ml amount of disinfecting agent in the range of about 12 ml to about 20 ml for each 1 m³ of the predetermined space, the propellant includes a mixture of nitrogen and carbon dioxide, the predetermined pressure is in the range of about 2 bar to about 14 bar, and the space having the predetermined volume includes a volume in the range of about 2 m³ to about 100 m³.

3. The system of claim 1 in which the predetermined volume of the container is about 125 ml, the predetermined amount of the disinfecting agent is about 24 ml, the predetermined pressure is in the range of about 2 bar to about 3 bar, and the space having the predetermined volume is about 2 m³.

4. The system of claim 1 in which the predetermined volume of the container is about 210 ml, the predetermined amount of the disinfecting agent is about 60 ml, the predetermined pressure is in the range of about 3 bar to about 4 bar, and the space having the predetermined volume is about 5 m³.

5. The system of claim 1 in which the predetermined volume of the container is about 520 ml, the predetermined amount of the disinfecting agent is about 120 ml, the predetermined pressure is in the range of about 3 bar to about 5 bar, and the space having the predetermined volume is about 10 m³.

6. The system of claim 1 in which the predetermined size of the container is about 800 ml, the predetermined amount of the disinfecting agent is about 300 ml, the predetermined pressure is in the range of about 4 bar to about 6 bar, and the space having the predetermined volume is about 25 m³.

7. The system of claim 1 in which the predetermined size of the container is about 1500 ml, the predetermined amount of the disinfecting agent is about 600 ml, the predetermined pressure is in the range of about 7 bar to about 10 bar, and the space having the predetermined volume is about 50 m³.

8. The system of claim 1 in which the predetermined size of the container is about 2500 ml, the predetermined amount of the disinfecting agent is about 1200 ml, the predetermined pressure is in the range of about 8 bar to about 14 bar, and the space having the predetermined volume is about 100 m³.

9. The system of claim 1 wherein the container and diffusion device and a combination of predetermined volume of the container, the predetermined amount of the disinfecting agent, and the predetermined pressure of the propellant are configured for a desired space.

10. The system of claim 1 in which the disinfecting agent includes a balanced mixture of acetic acid (CH₃CO₂H), hydrogen peroxide (H₂O₂), peracetic acid (CH₃CO₃H), and water (H₂O).

11. The system of claim 10 in which the disinfecting agent includes a mixture of about 5% peracetic acid (CH₃CO₃H), about 20% hydrogen peroxide (H₂O₂), about 5% acetic acid (CH₃CO₂H), and about 70% water (H₂O).

12. The system of claim 2 in which the mixture of nitrogen and carbon dioxide includes about 67% nitrogen and about 33% carbon dioxide.

13. The system of claim 2 in which the mixture of nitrogen and carbon dioxide includes about 75% nitrogen and about 25% carbon dioxide.

14. The system of claim 1 in which the fog of disinfecting agent disinfects at least 10⁶ pathogens in the air and surfaces of the space.

15. The system of claim 1 in which the fog of disinfecting agent disinfects at least 10⁵ pathogens in the air and surfaces of the space.

16. The system of claim 1 in which the fog of disinfecting agent disinfects at least 10⁴ pathogens in the air and surfaces of the space.

17. The system of claim 1 further including a bag disposed inside the container configured to store the disinfecting agent therein.

18. The system of claim 17 in which the bag is configured to prevent the disinfecting agent from reacting with the container.

19. The system of claim 1 in which the fog of disinfecting agent disinfects at least 10⁵ pathogens in the air and surfaces of the space.

20. A device for dispensing a disinfecting agent, the device comprising:
a container made of a non-oxidizing, non-elastic material of a predetermined volume in the range of about 125 ml to about 2500 ml configured to store a predetermined amount of a disinfecting agent in the range of about 12 ml to about 20 ml for each 1 m³ of a predetermined space and a propellant including carbon dioxide and nitrogen at a predetermined pressure in the range of about 2 bar to about 14 bar;
a diffusion device coupled to the container configured to nebulize a fog of the disinfecting agent into a space having a predetermined volume in the range of about 2 m³ to about 100 m³ and pathogens therein; and
wherein the container and the diffusion device and a combination of predetermined volume of the container, the predetermined amount of the disinfecting agent, and the predetermined pressure of the propellant are configured for the space such that the fog of disinfecting agent disinfects the pathogens in the air and surfaces of the space.

21. A method for dispensing a disinfecting agent, the method comprising:
providing a container made of a non-oxidizing, non-elastic material of a predetermined volume configured to store a predetermined amount of a disinfecting agent and a predetermined propellant at a predetermined pressure;
providing a diffusion device coupled to the device to nebulize a fog of disinfecting agent into a space having a predetermined volume and pathogens therein;
choosing a combination of the predetermined volume of the container, the predetermined amount of disinfecting agent, and the predetermined pressure of the propellant for the space such that the fog of disinfecting agent disinfects the pathogens in the space; and
placing the container and the diffusion device in the space and activating the diffusion device to dispense the fog of disinfecting agent into the space to effectively disinfect pathogens in the air and surfaces of the space.
